(19) United States
(12) Patent Application Publication
(10) Pub. No.: US 2017/0172460 A1
(43) Pub. Date: Jun. 22, 2017

(54) ENDOBRONCHIAL BRUSH DEVICE TO ESTIMATE SIZE OF AIRWAYS

(71) Applicant: SPIRATION, INC., Redmond, WA (US)

(72) Inventor: Hugo Xavier Gonzalez, Woodinville, WA (US)

(73) Assignee: Spiration, Inc. d.b.a. Olympus Respiratory America, Redmond, WA (US)

(21) Appl. No.: 15/300,424

(22) PCT Filed: Mar. 30, 2015
(86) PCT No.: PCT/US15/23400

§ 371 Date: Sep. 29, 2016

Related U.S. Application Data
(60) Provisional application No. 61/973,110, filed on Mar. 31, 2014.
(60) Provisional application No. 61/973,137, filed on Mar. 31, 2014.
(60) Provisional application No. 61/973,142, filed on Mar. 31, 2014.
(60) Provisional application No. 61/973,105, filed on Mar. 31, 2014.
(60) Provisional application No. 61/973,169, filed on Mar. 31, 2014.

(51) Int. Cl. A61B 5/107; A61B 5/08; A61B 1/04; A61B 1/267; A61B 90/00

(52) U.S. Cl. CPC A61B 5/1076; A61B 1/2676; A61B 90/06; A61B 5/1079; A61B 1/04; A61B 5/08; A61B 2090/061

(57) ABSTRACT

An airway measurement/estimation device may comprise a sizing shaft at least partially disposable with a bronchoscope. The sizing shaft may comprise a plurality of bristles of known lengths such that when the sizing shaft is inserted into a patient's airway, and viewed through a bronchoscope, the size or diameter of the airway may be approximated based at least in part on the size of the bristles in relation to the size of the airway.

### CROSS-REFERENCE TO RELATED APPLICATIONS

This application claims the benefit of U.S. Provisional Patent Application No. 61/973,110, filed Mar. 31, 2014, (entitled “ENDOBRONCHIAL BRUSH DEVICE TO ESTIMATE SIZE OF AIRWAYS”), the entire disclosure of which is hereby incorporated by reference. Any and all priority claims identified in the Application Data Sheet, or any correction thereto, are hereby incorporated by reference under 37 CFR 1.57.

### BACKGROUND OF THE INVENTIONS

### Field of the Inventions

The present technology generally relates to bronchial airway sizing.

### SUMMARY OF THE INVENTIONS

The systems, methods and devices described herein have innovative aspects, no single one of which is indispensable or solely responsible for their desirable attributes. Without limiting the scope of the claims, some of the advantageous features will now be summarized.

In some embodiments, an airway sizing device may comprise a sizing shaft, wherein the sizing shaft comprises a distal end and first and second bristles attached to the distal end of the sizing shaft. The first and second bristles may be different lengths. The sizing shaft may be at least partially disposable within at least part of a bronchoscope.

In some embodiments, the sizing shaft may comprise a plurality of bristles, some of which may be substantially similar in length to the first bristle and sonic of which may be substantially similar in length to the second bristle. The plurality of bristles may comprise bristles that are of a length different from both the first and second bristles. The bristles may be disposed substantially orthogonal to the sizing shaft, or the bristles may be disposed at a different angle, including an acute angle, relative to the sizing shaft. The bristles may be disposed at the same or different angles from each other, relative to the sizing shaft. The bristles may be disposed at generally a single position along a length of the sizing shaft, or may be disposed along some length of the sizing shaft. Bristles that are disposed close to the distal end of the sizing shaft may have lengths that are longer than the bristles disposed further from the distal end of the sizing shaft. The sizing shaft may comprise a flexing mechanism, which may include one or more of a ball joint, a spring, and/or a segment of the sizing shaft having increased flexibility.

In some embodiments, a method of measuring or estimating the size of an airway may comprise inserting a bronchoscope into an airway of a patient, inserting a sizing shaft into an airway of the patient, viewing at least part of the sizing shaft in the airway, and approximating a dimension of the airway based at least in part on the size of the airway compared to the size of at least one bristle. The sizing shaft may comprise a distal end and a first and second bristle attached to the distal end. The first and second bristles may be of different lengths. The dimension estimated may be a diameter of the airway. The sizing shaft may further comprise a plurality of bristles, wherein the plurality of bristles may have different lengths, some of which may be similar to the first bristle or the second bristle, or different from either the first or second bristle. In some embodiments the plurality of bristles may comprise three or more discrete categories of lengths of bristles. The method may further comprise inserting the sizing bristle device at least partially within the bronchoscope.

One benefit of a bronchoscope that comprises two or more bristles, wherein the two or more bristles are of two or more lengths, may be that it provides a user with a quick and accurate reading regarding the dimensions of an airway. In some embodiments, the user need only insert one bronchoscope into the patient's airway to determine the airway's dimension. In addition, the dimensions of the airway may be interpreted using a camera lens and visual images. This may be a more accurate method and/or results may be more easily obtained, as compared to other methods to determine the dimensions of an airway. For instance, some devices configured to estimate the diameter of an airway comprise an inflatable bladder or balloon wherein the amount that the balloon is inflated indicates the diameter of the airway. However, this device may require the user to determine the diameter of the airway based on feel, and may be subject to error. Other methods may require more than one device to be serially inserted into an airway to determine the diameter of the airway. This method may require more time, and may require more devices.

An airway sizing device can comprise a sizing shaft. The sizing shaft can include a distal end. In some embodiments, the sizing shaft includes a first bristle attached to the distal end. The sizing shaft can include a second bristle attached to the distal end. The first bristle can be a first length. The second bristle can be a second length. The first length can be different from the second length. The sizing shaft can be at least partially disposable within a lumen of a bronchoscope.

The sizing shaft can comprise a plurality of bristles. At least one of the plurality of bristles can be approximately the same length of the first bristle. The at least one of the plurality of bristles can be approximately the same length of the second bristle.

In some embodiments, the plurality of bristles can be a different length than either the first bristle or the second bristle. The first bristle and the second bristle can be disposed substantially orthogonal to the sizing shaft. The first bristle and the second bristle can be disposed at acute angles to the sizing shaft.

The first bristle and the second bristle can be disposed at different locations along the sizing shaft. The longer of the first bristle and the second bristle can be disposed closer to the distal end of the sizing shaft than the shorter of the first bristle and the second bristle. The sizing shaft can comprise a flexing mechanism.

The flexing mechanism can comprise a ball joint. The flexing mechanism can comprise a spring. The flexing mechanism can comprise two portions of the sizing shaft with two different degrees of flexibility.

A method of estimating the size of an airway can comprise inserting a bronchoscope into an airway of a patient. The method can comprise inserting a sizing shaft into an airway of the patient. The sizing shaft can comprise a distal end, a first bristle attached to the distal end, and a second bristle attached to the distal end. The first bristle can be a different length from the second bristle. The method can comprise viewing at least part of the sizing shaft in the airway. The method can comprise approximating a dimension of the airway based at least in part on the size of the airway as compared to at least one of the first bristle and the second bristle.

The dimension of the method of estimating the size of an airway can be a diameter. The sizing shaft of the method can comprise a plurality of bristles. The at least some of the plurality of bristles can have lengths substantially similar to the first bristle. Some of the plurality of bristles can have lengths substantially similar to the second bristle, and some of the plurality of bristles can have lengths that are not substantially similar to either the first bristle or the second bristle.

The sizing shaft of the method can further comprise three or more discrete lengths of bristles. The method can further comprise inserting the sizing shaft into at least part of the bronchoscope.

An airway sizing device can comprise a sizing shaft. The sizing shaft can comprise a distal end. The sizing shaft can comprise a first bristle attached to the sizing shaft at the distal end. The sizing shaft can comprise a second bristle attached to the sizing shaft at the distal end. The first bristle can be a first length. The second bristle can be a second length. The first length can be different from the second length. Both the first and the second bristle can emanate generally radially from the sizing shaft.

The first and second bristle can comprise one end attached to the sizing shaft while the other end is not attached to the sizing shaft. The first and second bristle can have a middle portion attached to the sizing shaft, and both ends of each bristle can be free to move. The sizing shaft can comprise a plurality of bristles. The plurality of bristles can comprise a generally cylindrical shape.

The first bristle can be a different color than the second bristle. The first bristle can be a different pattern or texture from the second bristle.

### BRIEF DESCRIPTION OF THE DRAWINGS

FIG. 1A is a side view of one embodiment of a bronchial airway sizing device.

FIG. 1B is a side view of another embodiment of a bronchial airway sizing device,

FIG. 1C is a side view of another embodiment of a bronchial airway sizing device.

FIG. 1D is a side view of another embodiment of a bronchial airway sizing device.

FIG. 2A is a side view of a bronchial airway sizing device illustrating a sizing shaft and its distal end.

FIG. 2B is a perspective view of an embodiment of a bronchial airway sizing device inserted into a first opening of a gauge.

FIG. 2C is a perspective view of an embodiment of a bronchial airway sizing device inserted into a second opening of a gauge.

FIG. 2D is a perspective view of an embodiment of a bronchial airway sizing device inserted into a third opening of a gauge.

FIG. 3A is a perspective view of an embodiment of a bronchial airway sizing device inserted into an airway.

FIG. 3B is a perspective view of the device of FIG. 3A inserted into another portion of an airway.

FIG. 4A is a side view of an embodiment of an embodiment of a bronchial airway sizing device coupled with a bronchoscope and inserted into an airway.

FIG. 4B is a side view of an embodiment of a bronchial airway sizing device with a flexibility mechanism.

FIG. 5A is a side view of an embodiment of a bronchial airway sizing device with a flexibility mechanism that comprises a coil spring.

FIG. 5B is a side view of an embodiment of a bronchial airway sizing device with a flexibility mechanism that comprises a ball joint.

### DETAILED DESCRIPTION

In the following detailed description, reference is made to the accompanying drawings, which form a part of the present disclosure. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and form part of this disclosure. For example, a system or device may be implemented or a method may be practiced using any number of the aspects set forth herein. In addition, such a system or device may be implemented or such a method may be practiced using other structure, functionality, or structure and functionality in addition to or other than one or more of the aspects set forth herein. Elements that are described as “connected,” “engaged,” “attached,” or similarly described, shall include being directly and/or indirectly connected, engaged, attached, etc. Alterations and further modifications of the inventive features illustrated herein, and additional applications of the principles of the inventions as illustrated herein, which would occur to one skilled in the art and having possession of this disclosure, are to be considered within the scope of the inventions.

Descriptions of unnecessary parts or elements may be omitted for clarity and conciseness, and like reference numerals refer to like elements throughout. In the drawings, the size and thickness of layers and regions may be exaggerated for clarity and convenience.

Features of the present disclosure will become more fully apparent from the following description and appended claims, taken in conjunction with the accompanying drawings. It will be understood these drawings depict only certain embodiments in accordance with the disclosure and, therefore, are not to be considered limiting of its scope; the disclosure will be described with additional specificity and detail through use of the accompanying drawings. An apparatus, system or method according to some of the described embodiments can have several aspects, no single one of which necessarily is solely responsible for the desirable attributes of the apparatus, system or method. After considering this discussion, and particularly after reading the section entitled “Detailed Description” one will understand how illustrated features serve to explain certain principles of the present disclosure.

It may be desirable to measure or approximate the size of an airway at a target location. In some cases, an approximation of the size of an airway can be used to select an appropriate treatment device for deployment into an airway. For example, valves or other medical devices of varying sizes may be available for deployment into an airway. In some applications, deployment of a device that is too large or too small for a given target location can result in undesirable treatment results. For example, deployment of a device that is too small for a given target location can results in a failure of the device to anchor or otherwise remain in place at the target location. In some cases, deployment of a device that is too large can result in damage to the device and/or to the airway at the target location. Deployment of a device that is too large can, in some instances, result in failure of the device to operate in a desired manner.

In some embodiments, an airway sizing bristle device comprises at least a handle at a proximal end, and sizing mechanism at the distal end. The handle may comprise a gripping portion, for which a user may grip or engage the sizing bristle device. The distal end of the sizing bristle device may comprise a sizing shaft and one or more bristles. The sizing bristle device may comprise a shaft or connector that connects the handle to the sizing mechanism at the distal end.

To use the bristle device one may insert the distal end into the patient's airway, The distal portion may then be located in the airway desired by the user, and the distal end of the bristle device may be disposed at or around the desired site in the airway. The user may use the distal end to determine or approximate the size of the airway, which may include the airway's diameter, or other cross-sectional or volume estimation. The bristle device may be delivered to the desired airway site via a working channel of a bronchoscope.

The distal end of the bronchoscope may comprise a camera, or other electronic device, that may provide the user a visual image of at least part of the bristle device and the desired site of the airway. The user may interpret information from the visual image to make a decision about which medical device (e.g., which size) to use in the patient's airway. In some embodiments the medical device to be used in the patient's airway includes a bronchial valve.

The distal end of the bristle device may comprise a sizing shaft and one or more bristles. In some embodiments, the sizing shaft has a generally elongated structure that is disposed or disposable in at least part of the bronchoscope. The sizing shaft may exit the distal end of the bronchoscope through an aperture (e.g., working channel). The sizing shaft may be extendable beyond the distal end of the bronchoscope by several centimeters or more. The width of the sizing shaft may be on the order of millimeters, although the width need not be uniform throughout the length of the shaft. The proximal end of the sizing shaft may be disposed or disposable near the handle of the bristle device. The sizing shaft may comprise a plastic or metal material. In some embodiments, the sizing shaft comprises a metal alloy such as Nitinol, and may have certain shape retaining and/or shape memory properties. In some embodiments, the sizing shaft may have a flexural, columnar, and/or torsional rigidity that allows the shaft to be bent but have a shape memory property. In some embodiments, the sizing shaft may be a flexible shaft such as a wire braid or coil which is flexible and has some torsional and/or columnar stiffness.

One or more bristles may be attached to the sizing shaft and be located at or near the distal end of the shaft. The bristles may comprise small fibers or small shafts, and may emanate generally radially from the sizing shaft. The bristles may comprise a generally cylindrical shape, or the cross-section of the bristles may be of a different shape. The bristles may be generally straight, although in some embodiments, when no external forces are acting on the bristles, the bristles may be straight or slightly bent. A first end of a bristle may attach to the sizing shaft, while a second end of a bristle may not be attached to the sizing shaft and may generally be configured to move. In some embodiments, the bristle is disposed through or next to the sizing shaft, such that a middle portion of the bristle attaches to the sizing shaft, and both the first and second ends of the bristle are free to move. The bristles may comprise various materials, which may include plastics or nylon, or other generally flexible materials that are suitable for medical use. The bristles may be of varying flexural strength. For example, one or more of the bristles may be soft and easily flexed in order to minimize irritation to the airway. The bristles may be sterilized or coated with a substance that minimizes the chances of infection in the airway.

In some embodiments, the configuration of the one or more bristles at the distal end of the sizing shaft may be varied. For instance, the number of bristles disposed on the sizing shaft may be increased or decreased depending on the configuration desired. As another example, the bristles may be attached generally to a single location along the length of the sizing shaft, or they may be distributed along some length of the sizing shaft. As another example, the bristles may be more flexible or less flexible, depending on the configuration desired. As another example, the length of the bristles or the distance from the free end of the bristle radially to the sizing shaft may vary depending on the configuration desired. In some embodiments, the one or more bristles disposed on the sizing shaft may not be uniform in size, shape, material, physical properties, disposition, or other properties that may be varied.

The bronchoscope may comprise at least a distal end, a middle section, and a proximal end. The distal end may comprise at least an aperture (e.g., working channel) for the sizing shaft and an aperture for an electronic device, which may comprise a camera and/or camera lens. In some embodiments, the distal end of the bronchoscope may comprise an aperture configured such that a sizing shaft may be disposed within at least part of the aperture. The aperture may be located at or near the distal end of the bronchoscope such that an axis of a sizing shall disposed within the aperture may be generally parallel to an axis of the bronchoscope. In some embodiments, the airway sizing device can be configured to fit within a catheter. The catheter can be sized and shaped to fit into and through a working channel or other lumen of the bronchoscope. At least part of the bronchoscope and the aperture may define a lumen that is disposed in at least part of the bronchoscope. In some embodiments, the lumen is in fluid communication with the aperture at the distal end of the bronchoscope and with a second aperture at or near the proximal end of the bronchoscope. In some embodiments, the sizing shaft may be disposable next to the bronchoscope, such that the sizing shaft does not substantially travel through a lumen of the bronchoscope.

The aperture for the camera may be adjacent the aperture for the sizing shaft. The camera may be disposed outside of the aperture or within the aperture or proximal of the aperture. The camera may be in operational communication with a viewing device, such that a user of the bronchoscope or the bristle device may observe visual images based on the information transmitted by the camera.

The proximal end of the bronchoscope may comprise a proximal end of the sizing shaft. The proximal end of the sizing shaft may at least partially protrude from the end of the bronchoscope and may comprise a feature through which a user may affect the position of the distal end of the sizing shaft.

The sizing shaft may comprise different variations depending on the specific needs of the user. Some of the variations may include different sizes, locations, and projections of the bristles. For instance, as a non-limiting example, the sizing shaft may comprise at least two different lengths of bristles. In some embodiments, the sizing shaft may comprise three different lengths of bristles, as measured from a free end of the bristle radially towards the sizing shaft. The bristles may be of a certain length, or the ends of the bristles may signify a certain distance from the center of the sizing shaft, or from one or more other bristles. The sizing shaft may comprise more or fewer lengths of bristles, and the scope of the inventions is not limited by the lengths of the bristles.

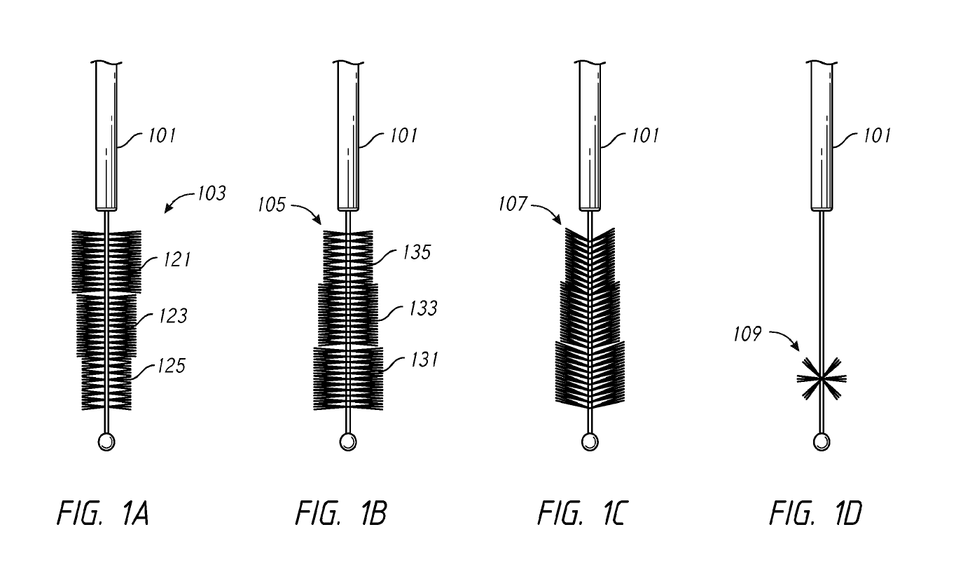

As illustrated in FIGS. 1A-1C, an airway measuring device can include one or more bristles 103 extending from or through a distal end of a sizing shaft 101. The one or more bristles 103 can have varying lengths (e.g., heights as measured orthogonally from the shaft 101). For example, one or more bristles 103 closest to the distal end of the shaft 101 can be shorter than one or more of the bristles 103 further from the distal end of the shaft 101. The lengths of the bristles 103 can vary on a stepped pattern, a sloped pattern, a parabolic pattern and/or some combination thereof along a length of the sizing shaft 101.

In some embodiments, as illustrated in in FIG. 1A, a first group of bristles 125, nearest the distal end of the sizing shaft 101, are shorter than a second group of bristles 123 further away from the distal end of the sizing shaft 101. The airway sizing device can include a third group of bristles 121 longer than the second group of bristles 123 and positioned further from the distal end of the sizing shaft 101 than the second group of bristles 123. For instance, as a non-limiting example, one or more bristles in the first group 125 disposed on the sizing shaft 101 may be a first length (e.g., 5 mm) as measured from the center of the sizing shaft 101 to a free end of a bristle. One or more bristles in the second group 123 may be disposed on the sizing shaft 101 proximal to the first group of bristles 125, and may have a second length (e.g., 6 mm) greater than the first length. One or more bristles of the third group 121 may be disposed on the sizing shaft 101 proximal the second group of bristles 123, and can have a third length (e.g., 7 mm) greater than the second length. Many variations of the lengths and the differences in lengths between the various groups of bristles are possible. For example, the shortest bristles can be less than 8 mm, less than 5 mm, less than 3 mm, and/or less than 1 mm in length. The longest bristles can be greater than 2 mm, greater than 4 mm, greater than 7 mm, and/or greater than 10 mm in length.

FIG. 1B illustrates some embodiments wherein the bristles 105 that are closest to the distal end of the sizing shaft 101 are longer than the bristles that are further away from the distal end of the sizing shaft 101. For example, a first bristle group 131 can be positioned closest to the distal end of the sizing shaft 101. A second bristle group 133 can be positioned adjacent the first bristle group 131, further from the distal end of the sizing shaft 101. In some embodiments, the device includes a third bristle group 135 positioned adjacent the second bristle group 133, further from the distal end of the sizing shaft 101. The bristles in the first bristle group 131 can be longer than the bristles in the second and third bristle groups. In some embodiments, the bristles in the second bristle group 133 are longer than the bristles in the third bristle group 135. Many variations are possible.

In some embodiments, the bristles of the first bristle group 131 may be approximately (e.g., within ±15%) 7 mm in length. In some embodiments, the bristles of the second bristle group 133 may be approximately 6 mm in length. In some embodiments, the bristles of the third bristle group 135 are approximately 5 mm in length.

One advantage to the configuration of FIG. 1B may be that all sizes of bristles may be seen using a camera that is proximal to the bristles. For instance, a camera located on the distal end of the bronchoscope may capture images of all three lengths of bristles, which may make it easier for a user to obtain a more accurate measurement and/or approximation of the size of the airway. The lengths of the bristles in various embodiments may be of generally discrete values. For instance, as a non-limiting example, some of the bristles may be approximately 5 mm in length, some may be 6 mm in length, and some may be 7 mm in length, with few to no bristles of lengths between these values. However, the actual length of the bristles need not be exactly any of these lengths, and may vary slightly. In some embodiments, the lengths of the bristles may be more continuous from one length to another. For instance, as a non-limiting example, some of the bristles may be between 4.5 mm and 5.5 mm in length, some may be between 5.5 mm and 6.5 mm in length, and some may be between 6.5 mm and 7.5 mm in length. However, the actual length of the bristles need not be exactly a certain length.

As illustrated in FIG. 1C, the bristles 107 disposed on the sizing shaft may not be orthogonal to the sizing shaft 101. For instance, as a non-limiting example, the bristles disposed on the sizing shaft 101 may be angled such that the free end of each bristle is more proximal than the location at which the bristle attaches to the shaft 101. Or, the bristles disposed on the sizing shaft 101 may be angled such that the free end of each bristle is distal of the point at which the bristle attaches to the shaft. Not all bristles need to be disposed at the same angle relative to the sizing shaft 101, and the scope of the inventions is not limited by the angle at which the bristles are disposed relative to the shaft 101. The bristles that are closest to the distal end of the sizing shaft 101 may be longer than the bristles that are further away from the distal end of the sizing shaft 101. Alternatively, the bristles that are further away from the distal end of the sizing shaft 101 may be longer than the bristles that are closest to the distal end of sizing shaft 101.

As illustrated in FIG. 1D the bristles 109 may be disposed on the sizing shaft 101 from generally a single location, and may not be substantially disposed along the sizing shaft 101. For instance, as a non-limiting example, one or more bristles may be disposed on a sizing shaft 101 with the bristles generally emanating from a single position along the length of the sizing shaft 101. The one or more bristles 109 attached to the sizing shaft 101 may be different lengths, as measured from the free end of the bristle to the sizing shaft 101. The bristles 109 may be generally disposed orthogonal to the sizing shaft 101. In some embodiments, the different length bristles are all intermixed circumferentially around the sizing shaft 101. In some embodiments, a single length of bristles, which may comprise more than one bristle, may generally occupy one or more sections circumferentially around the shaft 101, such that the different lengths of bristles are generally grouped together. In some embodiments, the bristles 109 disposed on the sizing shaft 101 may not be orthogonal relative to the shaft 101, and not all bristles need to be disposed at the same angle relative to the sizing shaft 101.

In some embodiments, the different length bristles may be identifiable using various indicators. For instance, bristles of a first length may be a first color, and bristles of a second length may be a second color, such that a user may be able to determine the length of a bristle viewed through a camera lens or other viewing device, based at least in part on the color of the bristle. In some embodiments, the bristles may comprise other indicators, such as patterns, textures, or other features. In some embodiments, different length bristles may be disposed at different angles relative to the sizing shaft, such that the length of the bristle, or the distance from a free end of the bristle to the shaft, may be generally determined based at least in part on the angle at which the bristle is disposed relative to the shaft.

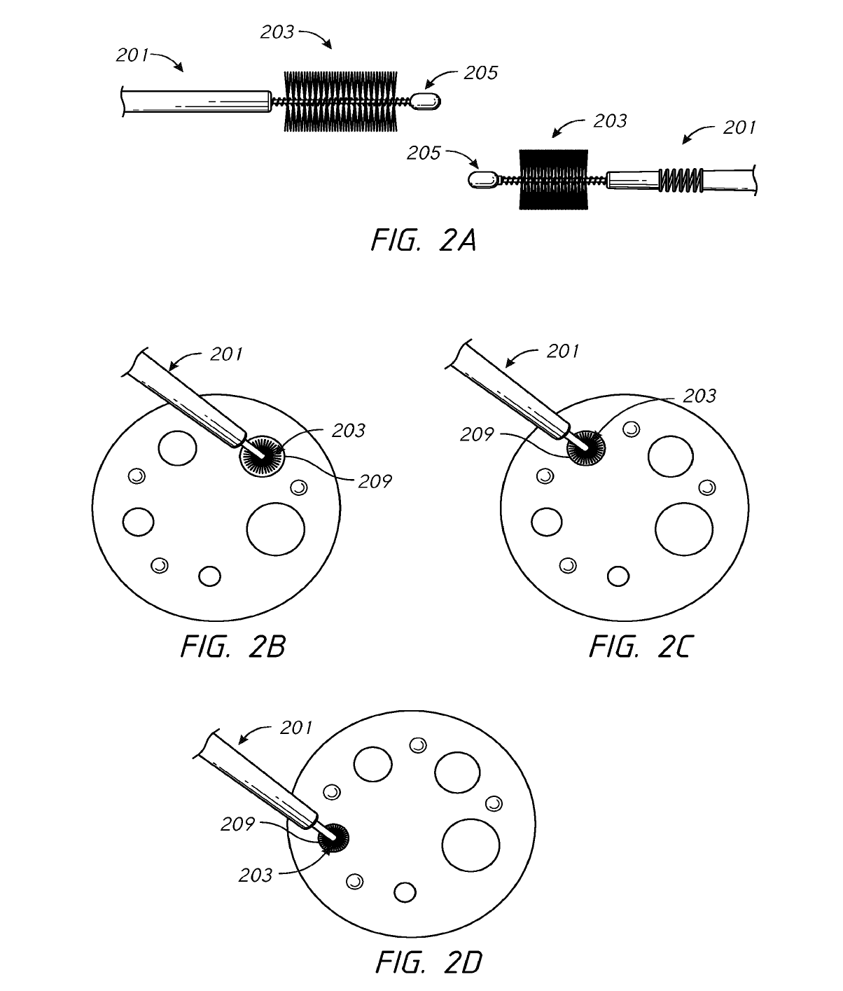

As shown in FIG. 2A, a sizing shaft 201 may comprise a distal end 205 and bristles 203 proximate the distal end 205. Bristles attached to a sizing shaft may be used to approximate the size of an opening. The opening may be a bronchial airway in a patient. As shown in FIG. 2B, if the bristles 203 are too small for the opening 209, there may be a space between the bristles 203 and the opening 209. Such a configuration may indicate to the user that the opening 209 is larger than the diameter of the bristles 203 and/or the distal end of the sizing shaft 201. As shown in FIG. 2C, if the opening 209 is approximately the same size as the diameter of the bristles 203 and/or the distal end of the sizing shaft 201, then the size of the bristles 203 may approximate the size of the opening 209. There will not be a gap and the bristles 203 will not be deformed by the opening 209. As shown in FIG. 2D, if the bristles 203 are too tight for the opening 209, then the opening 209 may be smaller than the size of the bristles 203. The bristles 203 will be visibly deformed or bent by the opening 209.

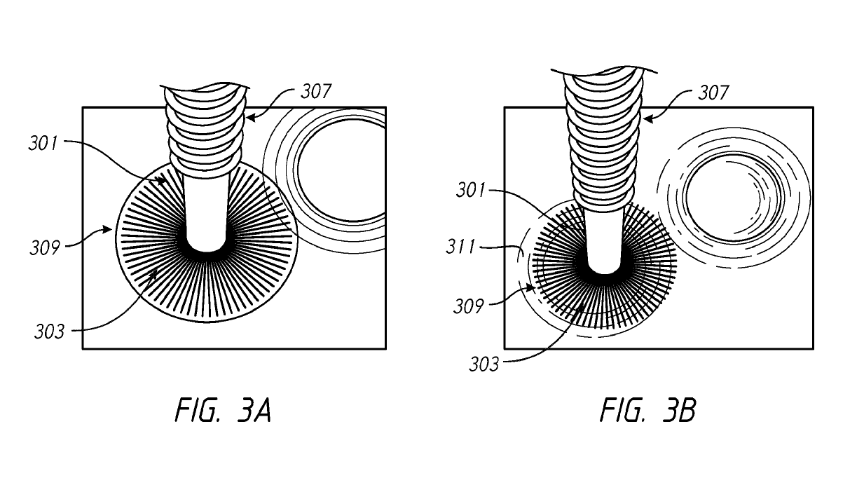

FIGS. 3A and 3B illustrate an embodiment of the device in an airway. Both figures show the sizing shaft 301, which is connected to a flexing mechanism 307. In these embodiments, the flexing mechanism 307 is a coil spring. The sizing shaft 301 may comprise any kind of flexing mechanism 307 that may help center and/or align the sizing shaft 301 relative to the airway 309. As shown in FIG. 3A, if the bristles 303 come close to or just touch the sides of an airway 309, then the size of the airway 309 may be approximated by the size of the bristles 303. There may be no visible deformation or bending of the bristles 303, which would otherwise suggest that the diameter of the bristles 303 are larger than the diameter of the airway 309. As shown in FIG. 3B, if there is space 311 between the bristles 303 and the sides of an airway 309, then the size of the airway 309 may be larger than the size of the bristles 303. The space 311 between the bristles 303 and the airway 309 can be seen in comparison with FIG. 3A, where there is no gap.

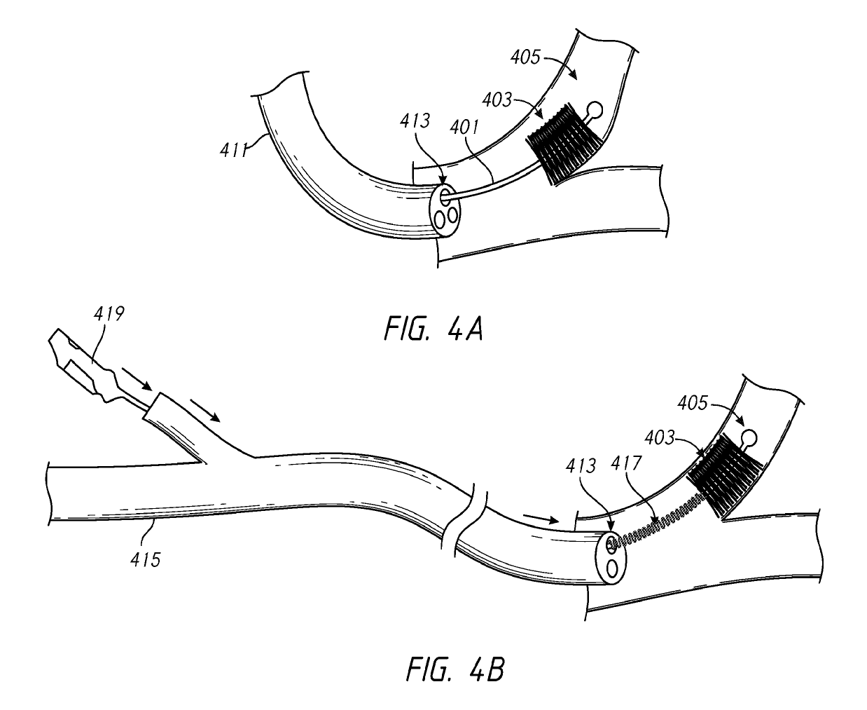

As illustrated in FIG. 4A, the device may be used in combination with a bronchoscope 411. The sizing shaft 401 may be disposed within a working channel 413 of bronchoscope 411. If the airway is straight at or near the point where the bristles 403 interact with the airway, a sizing shaft 401 with a generally uniform resistance to bending may suffice. In some cases, it may be advantageous or necessary to measure or approximate the size of an airway at or near a bend. In some such instances, the distal end of sizing shaft 405 may not center within the airway. If the airway curves at or near the point where the bristles 403 interact with the airway and the distal end of sizing shaft 405 is not centered, this can lead to some of the bristles being bent while also creating a gap between other bristles and the wall of the airway. For this reason, it may be advantageous for a sizing shaft 401 to comprise a mechanism that may help center and/or align sizing shaft 401 relative to the airway.

In some embodiments, the shaft 401, or sonic portion thereof, includes a curved portion. For example, the shaft 401 can include a curved portion at or near the distal end of the shaft 401. The curved portion of the shaft 401 can aid in the process of inserting/deploying the bristles 403 in a curved lumen. In some embodiments, the shaft 401 or some other portion of the device includes a visual marker to aid a user in determining the direction of curvature of the curved portion of the shaft 401. In some cases, the shaft 401 or some other portion of the device includes a guide wire configured to permit a user to curve a portion of the device into a curved lumen.

As illustrated in FIG. 4B the sizing shaft can comprise a flexing mechanism 417 that may help center and/or align the sizing shaft relative to the airway. Note that the flexing mechanism 417 can center the distal end of sizing shaft 405. The bristles 403 can now be properly used to determine the size of the curved airway. Movement of the device into and out of the airway may be controlled via a handle portion (e.g., handle of a catheter or other deployment mechanism) inserted through a proximal end 415 of the bronchoscope 411.

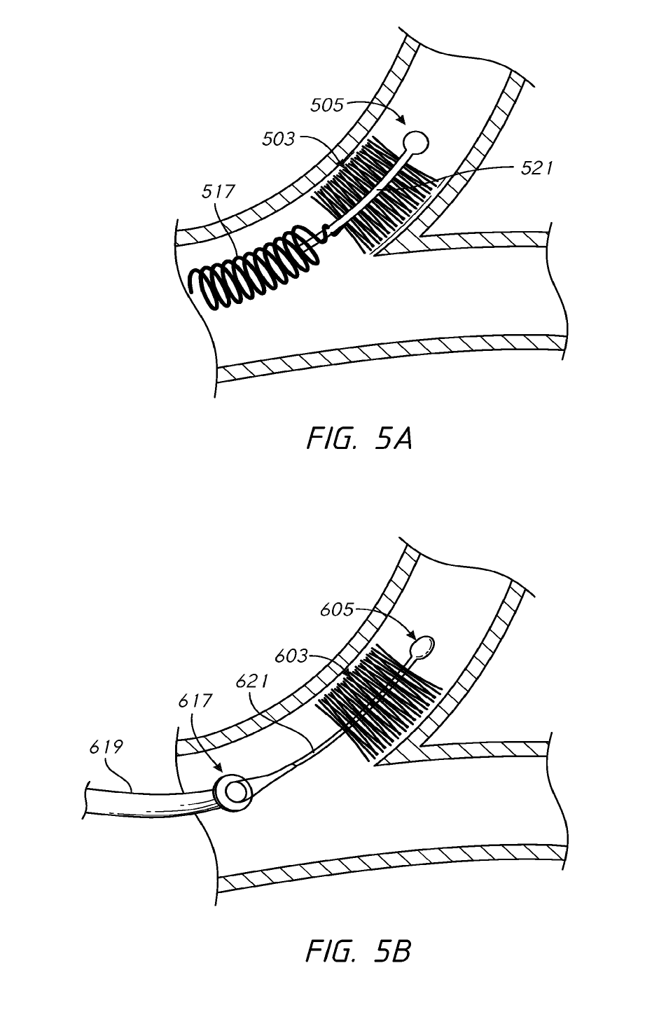

FIG. 5A illustrates a device with a flexing mechanism 517 in a curved airway. In some embodiments like this one, the flexing mechanism 517 comprises a spring. The spring may comprise a coil of metal or plastic or other material, such that a first portion (not shown) of the sizing shaft 505 attached to a proximal end of the flexing mechanism 517 may be capable of angularly deflecting relative to a second portion 521 of the sizing shaft.

FIG. 5B illustrates a device with a flexing mechanism 619 in a curved airway. In some embodiments like this one, the flexing mechanism 617 comprises a ball joint. The ball joint 617 may allow a first portion 619 of the sizing shaft to angularly deflect relative to a second portion 621 of the sizing shaft. The ball joint 617 may be disposed generally near the distal end of the sizing shaft 605, but proximal to one or more of the bristles 603.

In some embodiments, the flexing mechanism comprises a sizing shaft that has at least two portions having two different degrees of flexibility. For instance, the sizing shaft at or near the distal end may be more easily bent than another portion of the sizing shaft. A sizing shaft that is inserted into an airway may be more easily deflected and aligned with the airway if the distal end of the sizing shaft at or around the bristles may be easily deflected. This may be accomplished by altering the cross-sectional area or shape of the sizing shaft, or by altering the composition of the sizing shaft. The flexing mechanism may be controlled at the proximal end of the bristle sizing device such that the user may control the amount that the distal end of the device angularly deflects from the rest of the bristle sizing device. The bristle sizing device may comprise a pull wire or other remote controlling mechanism to control the amount of angular deflection of the sizing mechanism. The bristle sizing mechanism may be configured such that the user can rotate the sizing mechanism about the bristle sizing device's axis so that the sizing mechanism may angularly deflect in the direction desired by the user.

Terminology: Additional Embodiments

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein. Additionally, a person having ordinary skill in the art will readily appreciate, the terms “upper” and “lower” are sometimes used for ease of describing the figures, and indicate relative positions corresponding to the orientation of the figure on a properly oriented page, and may not reflect the proper orientation of the device as implemented.

Certain features that are described in this specification in the context of separate implementations also can be implemented in combination in a single implementation. Conversely, various features that are described in the context of a single implementation also can be implemented in multiple implementations separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. Additionally, other implementations are within the scope of the following claims. In some cases, the actions recited in the claims can be performed in a different order and still achieve desirable results.

In describing the present technology, the following terminology may have been used: The singular forms “a,” “an,” and “the” include plural referents unless the context clearly dictates otherwise. Thus, for example, reference to an item includes reference to one or more items. The term “ones” refers to one, two, or more, and generally applies to the selection of some or all of a quantity. The term “plurality” refers to two or more of an item. The term “about” means quantities, dimensions, sizes, formulations, parameters, shapes and other characteristics need not be exact, but may be approximated and/or larger or smaller, as desired, reflecting acceptable tolerances, conversion factors, rounding off, measurement error and the like and other factors known to those of skill in the art. The term “substantially” means that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide. Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3 and 4 and sub-ranges such as 1-3, 2-4 and 3-5, etc. This same principle applies to ranges reciting only one numerical value (e.g., “greater than about 1”) and should apply regardless of the breadth of the range or the characteristics being described. A plurality of items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. Furthermore, where the terms “and” and “or” are used in conjunction with a list of items, they are to be interpreted broadly, in that any one or more of the listed items may be used alone or in combination with other listed items. The term “alternatively” refers to selection of one of two or more alternatives, and is not intended to limit the selection to only those listed alternatives or to only one of the listed alternatives at a time, unless the context clearly indicates otherwise.

It should be noted that various changes and modifications to the presently preferred embodiments described herein will be apparent to those skilled in the art. Such changes and modifications may be made without departing from the spirit and scope of the inventions and without diminishing its attendant advantages. For instance, various components may be repositioned as desired. It is therefore intended that such changes and modifications be included within the scope of the inventions. Moreover, not all of the features, aspects and advantages are necessarily required to practice the present inventions. Accordingly, the scope of the present inventions is intended to be defined only by the claims that follow.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment. Conjunctions, such as “and,” “or” are used interchangeably and are intended to encompass any one element, combination, or entirety of elements to which the conjunction refers.

What is claimed is:

1. An airway sizing device comprising:
a sizing shaft comprising:
a distal end;
a first bristle attached to said distal end; and
a second bristle attached to said distal end;
wherein said first bristle is a first length and said second bristle is a second length and said first length is different from said second length; and
wherein said sizing shaft is at least partially disposable within a lumen of a bronchoscope.

2. The device of claim 1, wherein said sizing shaft further comprises a plurality of bristles, and wherein at least one of said plurality of bristles is approximately the same length of said first bristle.

3. The device of claim 2, wherein at least one of said plurality of bristles is approximately the same length of said second bristle.

4. The device of claim 1, wherein the sizing shaft further comprises a plurality of bristles, wherein said plurality of bristles are a different length than either said first bristle or said second bristle.

5. The device of claim 1, wherein said first bristle and said second bristle are disposed substantially orthogonal to said sizing shaft.

6. The device of claim 1, wherein said first bristle and said second bristle are disposed at acute angles to said sizing shaft.

7. The device of claim 1, wherein said first bristle and said second bristle are disposed at different locations along said sizing shaft.

8. The device of claim 1, wherein the longer of said first bristle and said second bristle is disposed closer to said distal end of said sizing shaft than the shorter of said first bristle and said second bristle.

9. The device of claim 1, wherein said sizing shaft further comprises a flexing mechanism.

10. The device of claim 1, wherein said flexing mechanism comprises one of a ball joint, a spring, or two portions of said sizing shaft with two different degrees of flexibility.

11. A method of estimating the size of an airway comprising:
inserting a bronchoscope into an airway of a patient;
inserting a sizing shaft into an airway of said patient;
wherein said sizing shaft comprises a distal end, a first bristle attached to said distal end, and a second bristle attached to said distal end; and
wherein said first bristle is a different length from said second bristle;
viewing at least part of said sizing shaft in said airway;
approximating a dimension of said airway based at least in part on the size of said airway as compared to at least one of said first bristle and said second bristle.

12. The method of claim 11, wherein said dimension is a diameter.

13. The method of claim 11, wherein said sizing shaft comprises a plurality of bristles.

14. The method of claim 13, wherein at least some of said plurality of bristles have lengths substantially similar to said first bristle, some of said plurality of bristles have lengths substantially similar to said second bristle, and some of said plurality of bristles have lengths that are not substantially similar to either said first bristle or said second bristle.

15. The method of claim 11, wherein said sizing shaft further comprises three or more discrete lengths of bristles.

16. The method of claim 11, further comprising inserting said sizing shaft into at least part of said bronchoscope.

17. An airway sizing device comprising:
a sizing shaft comprising:
a distal end;
a first bristle attached to the sizing shaft at said distal end; and
a second bristle attached to said sizing shaft at said distal end;
wherein said first bristle is a first length and said second bristle is a second length and said first length is different from said second length; and
wherein both the first and second bristle emanate generally radially from the sizing shaft.

18. The device of claim 17, wherein bath the first and second bristle have one end attached to the sizing shaft while the other end is not attached to the sizing shaft.

19. The device of claim 17, wherein both the first and second bristle have a middle portion attached to the sizing shaft, and both ends of each bristle are free to move.

20. The device of claim 17, wherein the sizing shaft further comprises a plurality of bristles, and the plurality of bristles comprise a generally cylindrical shape.

21. The device of claim 17, wherein the first bristle is a different color than the second bristle.

22. The device of claim 17, wherein the first bristle is a different pattern or texture from the second bristle.
